(19) United States
(12) Patent Application Publication
(10) Pub. No.: US 2022/0323018 A1
(43) Pub. Date: Oct. 13, 2022

(54) AUTOMATIC PREDICTION OF BLOOD INFECTIONS

(71) Applicant: RAMBAM MED-TECH LTD., Haifa (IL)

(72) Inventors: Anat SHROT, Nir Etzion (IL); Michael ROIMI, Nir Etzion (IL)

(21) Appl. No.: 17/615,919

(22) PCT Filed: Jun. 3, 2020
(86) PCT No.: PCT/IL2020/050620

§ 371 Date: Dec. 2, 2021

Related U.S. Application Data
(60) Provisional application No. 62/856,244, filed on Jun. 3, 2019.

(51) Int. Cl. A61B 5/00; G16H 50/20

(52) U.S. Cl. CPC A61B 5/7267; G16H 50/20

(57) ABSTRACT

A method for predicting a medical condition in a patient, the method comprising: receiving, with respect to each of a plurality of subjects, a plurality of clinical parameters, and an outcome indication with respect the said medical condition; applying to said plurality of clinical parameters one or more feature selection algorithms, to select a subset of said plurality of clinical parameters as the most relevant predictors; at a training stage, training a machine learning model on a training set comprising: (i) said relevant predictors with respect to each of said subjects, and (ii) labels associated with said outcome indication in said subject; and at an inference stage, applying said trained machine learning model to a target subset of said relevant predictors with respect to a target patient, to predict said medical condition in said target patient.

### CROSS-REFERENCE TO RELATED APPLICATIONS

This application claims benefit of U.S. provisional application Ser. No. 62/856,244 filed Jun. 3, 2019, and entitled “AUTOMATIC PREDICTION OF BLOOD INFECTIONS,” which is hereby incorporated herein by reference in its entirety.

### BACKGROUND

The invention relates to the field of automated medical diagnosis.

Nosocomial and similar hospital-acquired infections and diseases may account for significant morbidity and/or mortality in patients of hospitals and health care facilities such as intensive care units (ICUs).

For example, a nosocomial infection, e.g., a Bloodstream Infection (BSI) and/or a BSI, may be associated with decreased survival rates and/or an increased hospitalization period and/or ICU stay length. A crude mortality of patients suffering from BSI is above 30%.

Numerous studies have suggested that early recognition, appropriate antibiotic treatment, and/or removal of a source of BSI may be associated with a significant reduction of morbidity and/or mortality. However, an ability of physicians to predict whether an infection is accompanied and/or caused by a nosocomial infection, e.g., a primary BSI and/or secondary BSI, may be limited.

It may be advantageous to provide an automatic, e.g., machine learning (machine learning) based, algorithm and/or prediction model that could predict an infection, e.g., a hospital-acquired BSI, by using routinely collected easily accessed data.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in an embodiment, a system for predicting a medical condition in a patient, the system comprising at least one hardware processor; and a non-transitory computer-readable storage medium having stored thereon program instructions, the program instructions executable by the at least one hardware processor to: receive, with respect to each of a plurality of subjects, a plurality of clinical parameters, and an outcome indication with respect the said medical condition, apply to said plurality of clinical parameters one or more feature selection algorithms, to select a subset of said plurality of clinical parameters as the most relevant predictors, at a training stage, train a machine learning model on a training set comprising: (i) said relevant predictors with respect to each of said subjects, and (ii) labels associated with said outcome indication in said subject, and at an inference stage, apply said trained machine learning model to a target subset of said relevant predictors with respect to a target patient, to predict said medical condition in said target patient.

There is also provided, in an embodiment, a method for predicting a medical condition in a patient, the method comprising: receiving, with respect to each of a plurality of subjects, a plurality of clinical parameters, and an outcome indication with respect the said medical condition; applying to said plurality of clinical parameters one or more feature selection algorithms, to select a subset of said plurality of clinical parameters as the most relevant predictors; at a training stage, training a machine learning model on a training set comprising: (i) said relevant predictors with respect to each of said subjects, and (ii) labels associated with said outcome indication in said subject; and at an inference stage, applying said trained machine learning model to a target subset of said relevant predictors with respect to a target patient, to predict said medical condition in said target patient.

There is further provided, in an embodiment, a computer program product comprising a non-transitory computer-readable storage medium having program code embodied therewith, the program code executable by at least one hardware processor to: receive, with respect to each of a plurality of subjects, a plurality of clinical parameters, and an outcome indication with respect the said medical condition; apply to said plurality of clinical parameters one or more feature selection algorithms, to select a subset of said plurality of clinical parameters as the most relevant predictors; at a training stage, train a machine learning model on a training set comprising: (i) said relevant predictors with respect to each of said subjects, and (ii) labels associated with said outcome indication in said subject; and at an inference stage, apply said trained machine learning model to a target subset of said relevant predictors with respect to a target patient, to predict said medical condition in said target patient.

In some embodiments, the medical condition is a bloodstream infection (BSI), and said relevant predictors are selected from the group consisting of: blood urea nitrogen parameters, mean arterial pressure parameters, bilirubin parameters, blood pressure parameters, hospitalization duration parameters, body temperature parameters, neutrophils count parameters, blood oxygen saturation parameters, lymphocyte count parameters, anion gap parameters, and partial pressure of oxygen parameters.

In some embodiments, the medical condition is extubation failure risk and said relevant predictors are selected from the group consisting of: sedative drug dosage parameters prior to extubation, mean alveolar pressure parameters, hemodynamic parameters, respiratory parameters, heart rate parameters, respiratory rate parameters, and arterial blood pressure parameters.

In some embodiments, the medical condition is mortality risk within a specified time period, and said relevant predictors are selected from the group consisting of: hemodynamic parameters, respiratory parameters, heart rate parameters, respiratory rate parameters, and arterial blood pressure parameters, patient medical history, bilirubin parameters, hemoglobin parameters, red blood cell indices, glucose parameters, creatinine parameters, and albumin parameters.

In some embodiments, the patient medical history comprises prior medical diagnoses of at least some of: ischemic heart disease, congestive heart failure, chronic obstructive pulmonary disease, chronic renal failure, end-stage renal disease, diabetes without target organ damage, diabetes with organ damage, acute leukemia, chronic leukemia, lymphoma, multiple myeloma, human immunodeficiency virus infection, malignancy, cirrhosis, cerebral vascular accident, transient ischemic attack, and dementia.

In some embodiments, the relevant predictors further include gastro-intestinal function parameters selected from the group consisting of: defecation frequency during the preceding 24, 48, 72 and 96 hour periods; total time without defecations during the preceding 24, 48, 72 and 96 hour periods; vomiting frequency; evidence of the amount of gastric residual volume; gastric and intestinal acidity; and intra-abdominal pressure (TAP).

In some embodiments, the plurality of clinical parameters further comprises clinical data monitored in connection with hospital admission selected from the group consisting of: body temperature; hemodynamic and respiratory parameters; heart rate; systolic blood pressure; diastolic blood pressure; mean arterial pressure; urine output; respiratory rate; pulse oximetry O2 saturation; timing, duration, and dosage of intravenous fluids; diuretics; vasopressor; antibiotic treatment; total parenteral nutrition; enteral nutrition; continuous renal replacement therapy and dialysis; presence and timing of indwelling catheters; surgeries during admission; duration of hospitalization and ICU stay; use of glucocorticoids; and chemotherapy.

In some embodiments, the feature selection algorithms comprise at least an extreme gradient boosting algorithm.

In some embodiments, the machine learning model comprises a plurality of classification algorithms selected from the group consisting of: linear discriminant analysis (Ida), classification and regression trees (cart), It-nearest neighbors (knn), support vector machine (svm), logistic regression (glm), random forest (if), generalized linear models (glmnet), naive Bayes (nb), and extreme gradient boosting.

In some embodiments, the applying comprises applying each of said plurality of classification algorithms to said target subset to obtain a plurality of corresponding predictions, and wherein a final prediction of said medical condition is based, at least in part, on a weighted soft voting of all of said plurality of predictions.

In some embodiments, the soft voting is based, at least in part, on a confidence score associated with each of said plurality of predictions.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 shows a schematic illustration of an exemplary system for the detection, prediction, and/or diagnosis of medical conditions, according to an embodiment of the present disclosure;

FIG. 2 shows a flowchart illustrating an exemplary method for the detection, prediction, and/or diagnosis of medical conditions, according to an embodiment of the present disclosure; and

FIGS. 3A-4B illustrate experimental results of an exemplary method for the detection, prediction, and/or diagnosis of medical conditions, according to an embodiment of the present disclosure.

### DETAILED DESCRIPTION

Described herein are a system, method, and computer program product for generating automated models for the detection, prediction, and/or diagnosis of medical conditions based, at least in part, on routinely-collected medical and/or clinical data as an input.

In some embodiments, the present disclosure provides for analyzing medical and/or clinical data, to obtain a set of predictive features with respect to a specified medical condition and/or disease.

In some embodiments, the set of predictive feature may then be used as a training set to train a machine learning model. In some embodiments, a trained machine learning model of the present disclosure may be configured to predict a specified medical condition and/or disease.

The present disclosure will discuss extensively an embodiment of the present disclosure configured to predict blood stream infections in subjects. However, the present disclosure may be equally suitable and effective for predicting other and/or additional medical conditions and/or syndromes and/or diseases, including, but not limited to, extubation failure and mortality probability.

The term “blood stream infection” (BSI), or “bacteremia” is used herein to mean the presence of bacteria in a subject's blood system. BSI may or may not have any discernable symptoms prior to a successful diagnosis thereof. Symptoms of BSI include, but are not limited to fever, rapid heart rate, shaking chills, low blood pressure, gastrointestinal symptoms, such as but not limited to abdominal pain, nausea, vomiting, and diarrhea, rapid breathing, and/or confusion. If severe enough, BSI can lead to sepsis, severe sepsis and possible septic shock. Accordingly, the term BSI, as used herein, includes non-septic bacterial infection of the blood as well as septic bacterial blood infections. In some embodiments, the BSI that is treated or tested for is not sepsis, severe sepsis or septic shock. In other embodiments, the BSI that is treated or tested for is sepsis, severe sepsis or septic shock.

In some embodiments, an automated BSI infection detection and/or prediction algorithm of the present disclosure may be configured to identify patients who are at a high risk of having BSI and/or any other bacterial infection. Such observations may translate into meaningful diagnostic and/or therapeutic steps, aimed at treating patients for BSI, and at identifying and/or controlling its source.

In some embodiments, the present algorithm may be particularly useful in the context of bacterial infections contracted during hospitalization and/or within a hospital and/or similar medical facility environment.

Experimental results of the present algorithm show that it is possible to identify patients at high risk of BSI from 24 to 72 hours before blood cultures become positive, by using readily available data, e.g., which may be independent of any subjective clinical judgment and/or cost efficient.

In some embodiments, early identification of BSI, e.g., of a patient with a high likelihood of BSI, may be advantageous for a plurality of reasons, e.g., as follows:
Early identification may help and/or assist a physician to modify and/or change a management of a patient, e.g., by initiating and/or modifying an empiric antibiotic treatment, for example, since not all antibiotics are equally active against BSIs, even if a culprit pathogen is in vitro susceptible;
early identification may cause clinicians to be more inclined to perform additional diagnostic tests in order to find a focus of the BSI;
early identification may enable clinicians to eliminate a potential source of BSI and/or BSI, e.g., by withdrawing a potentially infected central venous line, draining an abscess and/or debriding an infected wound; and
early identification may enable clinicians to avoid unnecessary workup for other causes of fever in ICU patients, e.g., if a likelihood of BSI is deemed to be high.

In some embodiments, the present disclosure may be equally effective in predicting extubation failure risk in patients. In some embodiments, the present disclosure may provide for creating a machine learning model which predicts a risk of extubation failure in a patient. In some embodiments, primary predictor features for extubation failure risk are:
Cumulative dosage of sedating drugs during the 24, 48 and 72 hours prior to extubation;
ventilator measurements, including average and/or maximal minus average values of the mean alveolar pressure during the 24, 48 and 72 and 5 days prior to extubation; and/or
Time series analyses representing changing patterns of the hemodynamic and respiratory system:
   heart rate,
   respiratory rate, and/or
   arterial blood pressure.

In some embodiments, additional and/or other clinical parameters may be considered as primary predictors in the case of extubation failure risk prediction, including, but not limited to:
Gastro-intestinal function parameters:
   Defecation frequency during the 24, 48, 72 and 96 hours prior to the index time for outcome prediction,
   total time without defecations during these periods,
   vomiting frequency,
   evidence of the amount of gastric residual volume,
   gastric and intestinal acidity, and/or
   intra-abdominal pressure (TAP).

In some embodiments, the present disclosure may be equally effective in predicting mortality risk in patients within a specified time period from an index day, e.g., 30-days mortality risk. In some embodiments, the present disclosure may provide for creating a machine learning model which predicts a probability of death in a patient within a specified period from an index day, e.g., 30 days. In some embodiments, primary predictor features for 30 day mortality risk are:
Time series analyses representing changing patterns of the hemodynamic and respiratory system:
   heart rate,
   respiratory rate, and/or
   arterial blood pressure;
medical history of the patient;
patient age;
changes in laboratory measurement patterns of:
   bilirubin,
   hemoglobin,
   red blood cell indices,
   glucose,
   creatinine, and/or
   albumin.

In some embodiments, additional and/or other clinical parameters may be considered as primary predictors in the case of 30 day mortality risk prediction, including, but not limited to:
Gastro-intestinal function parameters:
   Defecation frequency during the 24, 48, 72 and 96 hours prior to the index time for outcome prediction,
   total time without defecations during these periods,
   vomiting frequency,
   evidence of the amount of gastric residual volume,
   gastric and intestinal acidity, and/or
   intra-abdominal pressure (IAP).

In some embodiments, the methods described herein involve two main steps: feature selection and machine learning-based classification.

In various embodiments, systems and methods of the present disclosure can execute machine learning algorithms to perform data mining, pattern recognition, intelligent prediction, and other artificial intelligence procedures, such as for enabling diagnostic predictions based on clinical data.

In some embodiments, an algorithm of the present disclosure may be configured to detect and/or predict symptoms of BSI prior to detection of the presence of bacteria in the patient's blood system. In some embodiments, the patient may be assessed by the present algorithm prior to the onset of any detectable symptoms of BSI, such as prior to there being detectable levels of bacteria in the patient's blood system. In some embodiments, the patient does not have detectable symptoms of any type of sickness or condition. In some embodiments, the patient has an injury, condition, or wound that puts the patient at risk of developing BSI, such as having a viral or bacterial infection, such as but not limited to urinary tract infection, meningitis, pericarditis, endocarditis, osteomyelitis, and infectious arthritis, having or developing bronchitis, undergoing a medical surgical or dental procedure, having an open wound or trauma, such as but not limited to a wound received in combat, a blast injury, a crush injury, a gunshot wound, an extremity wound, suffering a nosocomial infection, having undergone medical interventions such as central line placement or intubation, having diabetes, having HIV, undergoing hemodialysis, undergoing organ transplant procedure (donor or receiver), receiving a glucocorticoid or any other immunosuppressive treatments, such as but not limited to calcineurin inhibitors, mTOR inhibitors, IMDH inhibitors and biological or monoclonal antibodies.

In some embodiments, the patient does not have a condition that puts the patient at risk of developing BSI, prior to application of the methods described herein. In other embodiments, the patient has a condition that puts the patient at risk of developing BSI.

In some embodiments, an automated infection prediction algorithm of the present disclosure may be configured to differentiate between BSI and other infections and/or noninfectious inflammatory processes. According to this embodiment, the infection prediction algorithm may be configured to provide an indication of growth of any pathogen in at least one blood culture of a patient, e.g., which may be collected at a medical center.

In some embodiments, a growth of pathogens which likely represent contamination rather than true infection may not be considered as BSI. Such pathogens may include, e.g., coagulase-negative staphylococci, Corynebacterium species, Bacillus species, Diphteroides, Aerococcus, and/or Propionibacterium.

In some embodiments, the plurality of medical parameters may include, for example, at least one of: Demographic details, underlying medical conditions, vital signs, laboratory measurements, e.g., including microbiologic data, fluid balance, chronic medications, procedures during an admission, a timing of surgeries, dosage and/or duration of pharmacologic treatments, and a registry of all fatalities.

In some embodiments, the present algorithm may include preprocessing the dataset and/or determining new features based on the collected “raw” data. For example, a plurality of measurements of a specific parameter taken from a patient over a sequence of time periods may be combined and represented by a single parameter, which may be added to the dataset.

In some embodiments, the presnet algorithm may be configured to process the dataset by implementing machine learning algorithms and/or techniques, e.g., for predicting a probability of an infection, the machine learning algorithms including, for example, an ensemble of techniques such as Random Forest (random forest) and/or boosting.

In some embodiments, random forest and/or boosting ensemble techniques may include combining several learners, e.g., decision trees having comparatively weak performances when used independently, for example, through averaging and/or hard voting results from the decision trees, e.g., to create a single strong learner that can make accurate predictions.

In some embodiments, boosting ensemble techniques and/or algorithms may include training a set of learners, e.g., decision trees, added sequentially and/or one after another, where later learners are configured to focus on and/or correct mistakes and/or errors of earlier learners and to update their weights accordingly. Learners may be added until no further improvements can be made, e.g., according to a gradient descent method.

In some embodiments, an output of the set of learners, e.g., prediction results, may be combined to determine a combined prediction, e.g., by scoring the set of learners and averaging their results using a weighted average approach. For example, determining the combined prediction may provide an accurate predictive force for a wider range of input data, e.g., reducing both a bias and a variance of the decision trees.

In some embodiments, a boosting ensemble may utilize an Extreme Gradient Boosting (XGBoost) technique and/or algorithm, e.g., which may include an ensemble of gradient boosted decision trees, for example, designed for computational speed and/or model performance. The XGBoost technique may support implementation of Gradient Boosting algorithms (also referred to as “gradient boosting machine”), Stochastic Gradient Boosting, and/or Regularized Gradient Boosting.

For example, the XGBoost technique may enable an efficient training of decision trees, for example, by allowing parallelization of tree construction using all available Central processing Unit (CPU) cores during a training period.

In some embodiments, a random forest may include an ensemble of decision trees. A random forest, e.g., configured to decrease a variance of the decision trees, may be configured to create several decision trees, e.g., up to thousands, and to train each decision tree independently on a different random sample of the dataset, e.g., according to a Bootstrap Aggregation (bagging) technique and/or algorithm.

In some embodiments, instead of considering all features while splitting a node of a decision tree, a random forest considers for each decision tree only a subset of all features and selects a best feature out of the subset. An output may be determined by averaging prediction results from the decision trees.

In some embodiments, an ensemble of random forest and/or boosting techniques such as XGBoost may be trained, e.g., independently, on the dataset.

In some embodiments, after a training stage, a target patient's data may be preprocessed and used at an inference stage, for example, including implementing the trained ensemble on the new data. A result of all random forest and/or boosting techniques of the trained ensemble may be averaged, e.g., to determine accurate predictions to the new patients.

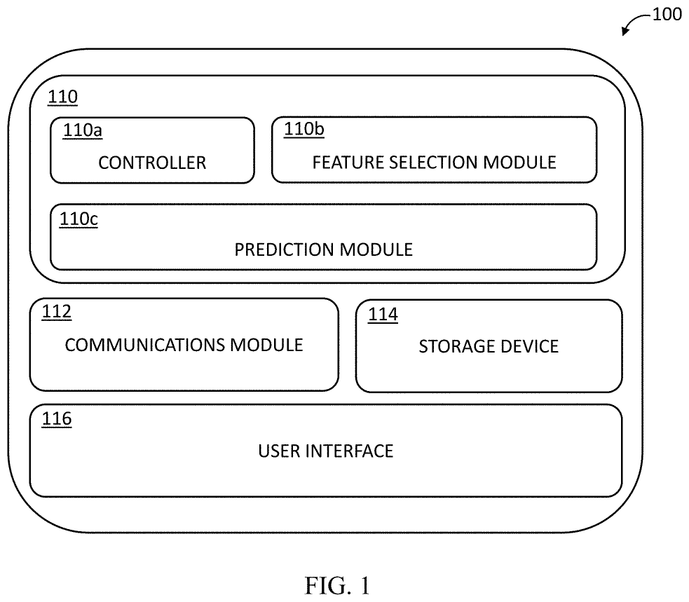

Reference is now made to FIG. 1, which is a schematic illustration of an exemplary system 100 for infection prediction. The various components of system 100 may be implemented in hardware, software or a combination of both hardware and software. System 100 as described herein is only an exemplary embodiment of the present invention, and in practice may have more or fewer components than shown, may combine two or more of the components, or a may have a different configuration or arrangement of the components.

In some embodiments, system 100 may include a processor 110, a controller 110a, a feature selection module 110b, a prediction module 110c, a communications module 112, a memory storage device 114, and/or a user interface 116. system 100 may store in a non-volatile memory thereof, such as storage device 114, software instructions or components configured to operate a processing unit (also “hardware processor,” “CPU,” or simply “processor), such as processor 110. In some embodiments, the software components may include an operating system, including various software components and/or drivers for controlling and managing general system tasks (e.g., memory management, storage device control, power management, etc.) and facilitating communication between various hardware and software components.

In some embodiments, non-transient computer-readable storage device 114 (which may include one or more computer readable storage mediums) is used for storing, retrieving, comparing, and/or annotating data and/or features. Data frames may be stored on storage device 114 based on one or more attributes, or tags, such as a time stamp, or a user-entered label, to name a few.

In some embodiments, communications module 112 may connect system 100 to a network, such as the Internet, a local area network, a wide area network and/or a wireless network. Communications module 112 facilitates communications with other external information sources and/or devices over one or more external ports and includes various software components for handling data received at system 100.

In some embodiments, user interface 116 may include circuitry and/or logic configured to interface between system 100 and at least one user of system 100. User interface 116 may be implemented by any wired and/or wireless link, e.g., using any suitable, Physical Layer (PHY) components and/or protocols.

In some embodiments, processor 110 may include controller 110a, feature selection module 110b, and/or prediction module 110c.

In some embodiments, controller 110a may be configured to perform and/or to trigger, cause, control and/or instruct system 100 to perform one or more functionalities, operations, procedures, and/or communications, to generate and/or communicate one or more messages and/or transmissions, and/or to control feature selection module 110b, prediction module 110c, communications module 112, memory storage device 114, user interface 116, and/or any other module and/or component of system 100.

In some embodiments, feature selection module 110b may be configured to receive as an input a plurality of medical features and/or parameters, for example, from communications module 112, memory storage device 114, and/or user interface 116, and to provide as an output a selected subset of the plurality of features, e.g., according to at least one criteria.

In some embodiments, prediction module 110c may be configured to receive as an input a plurality of features and/or parameters, for example, from feature selection module 110b and/or from any other component, and to provide as an output a prediction according to the plurality of features and/or parameters.

In some embodiments, controller 110a may be configured to cause system 100 to implement a solution for infection prediction, e.g., as described below.

In some embodiments, controller 110a may be configured to cause communications module 112 to receive a plurality of medical parameters relating to patients.

In some embodiments, controller 110a may be configured to preprocess the plurality of medical parameters to determine a plurality of medical, e.g., preprocessed, features.

In some embodiments, controller 110a may be configured to cause feature selection module 110b to select from the plurality of medical features a set of features based on a first machine learning boosting ensemble, e.g., an XGBoost.

In some embodiments, controller 110a may be configured to cause prediction module 110c to train a machine learning prediction ensemble on the set of features, e.g., to predict an infection of at least one patient.

In some embodiments, the machine learning prediction ensemble may include at least one second machine learning boosting ensemble, e.g., an XGBoost, and at least one random forest ensemble.

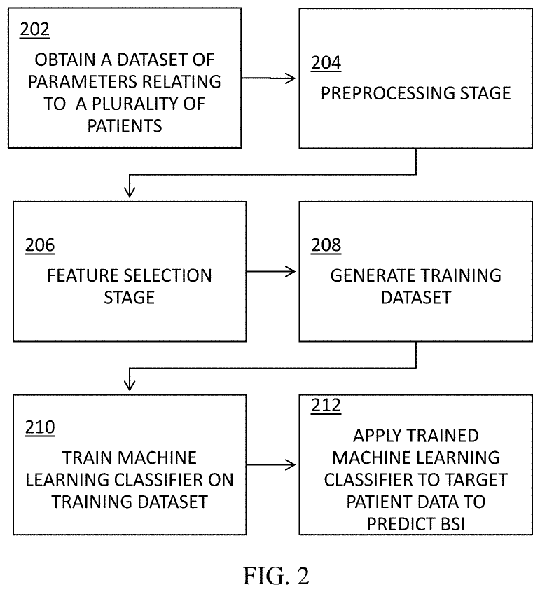

Reference is now made to FIG. 2, which is a flowchart illustrating exemplary method of infection prediction, according to certain embodiments of the present disclosure.

At a step 202, an exemplary system for detecting and/or predicting infection, such as system 100 in FIG. 1, may be configured to receive, obtain, and/or otherwise having received or obtained a dataset comprising a plurality of parameters relating to a plurality of patients. In some embodiments, these parameters may comprise one of more of factors, biomarkers, clinical parameters, and/or other parameters and components.

In some embodiments, such dataset may be constructed from medical data gathered from computerized database systems of medical centers, e.g., from one or more surgical, trauma-surgical, and medical ICUs.

In some embodiments, data gathered from the medical centers may include a plurality of medical parameters, e.g., demographic details, underlying medical conditions, vital signs, laboratory measurements, e.g., including microbiologic data, fluid balance, chronic medications, procedures during an admission, a timing of surgeries, dosage and/or duration of pharmacologic treatments, and/or a registry of all fatalities.

In some embodiments, the dataset may comprise at least some of the following:
Background demographic data: age, gender, ethnicity, weight, and date of admission of a patient.
Background clinical data: Prior medical diagnoses of ischemic heart disease, congestive heart failure, chronic obstructive pulmonary disease, chronic renal failure, end-stage renal disease, diabetes without target organ damage, diabetes with organ damage, acute leukemia, chronic leukemia, lymphoma, multiple myeloma, human immunodeficiency virus infection, malignancy, cirrhosis, cerebral vascular accident, transient ischemic attack, dementia, prior surgery, and timing of surgery in relation to ICU admission.
   Use of immunosuppressive drugs: Infliximab, adalimumab, etanercept, golimumab, anakinra, ustekinumab, tocilizumab, cyclosporine, tacrolimus, azathioprine, methotrexate, lenalidomide, and pomalidomide.
   Use of glucocorticoids.
   Chemotherapy.
Recorded clinical parameters from an admission to an ICU until a sampling time: Hourly temperature, hemodynamic and respiratory monitoring: Heart rate (HR), systolic blood pressure (SBP), diastolic blood pressure (DBP), mean arterial pressure (MAP), urine output, respiratory rate (RR), pulse oximetry O₂ saturation, and temperature.
   Laboratory assessment of the patient: Arterial blood gases that include pH, bicarbonate, lactate, Pco₂, PaO₂, base excess, sodium, chloride, potassium, magnesium, calcium, and glucose; renal function and/or electrolytes at least once daily: creatinine, blood urea nitrogen, sodium, potassium, chloride, phosphor, magnesium, uric acid; complete blood counts and leukocytes differential; coagulation tests and liver function tests when available, e.g., typically not measured daily; timing, duration, and dosage of intravenous fluids, diuretics, vasopressor, antibiotic treatment, total parenteral nutrition (TPN), enteral nutrition, continuous renal replacement therapy (CRRT) and dialysis.
   Presence and timing of indwelling catheters, which may include orotracheal tubes, tracheostomy tubes, central venous lines, dialysis catheters, and arterial line catheters.
   Surgeries during the admission.
   Duration of hospitalization and/or ICU stay prior to the sampling time.
   For ventilated patients, a recorded respiratory rate (RR), FIo₂ positive end-expiratory pressure (PEEP) and minute ventilation (MV).

In some embodiments, levels of the clinical parameters can be assayed, detected, measured, and/or determined in a sample taken or isolated from a patient. In some embodiments, examples of clinical parameters of a patient include, but are not limited to any one or more of gender, age, injury-related data (e.g., date of injury, location of injury, mechanism of injury, wound depth, wound surface area, associated injuries, type of wound closure, success of wound closure), requirement for transfusion, total number of blood products transfused, amount of whole blood cells administered to the patient, amount of red blood cells (RBCs) administered to the patient, amount of packed red blood cells (pRBCs) administered to the patient, amount of platelets administered to the patient, level of total packed RBCs, Injury Severity Score (ISS), Abbreviated Injury Scale (AIS) of head, AIS of abdomen, AIS of chest (thorax), Acute Physiology and Chronic Health Evaluation II (APACHE II) score, presence of critical colonization (CC) in a sample from the patient, presence of traumatic brain injury, severity of traumatic brain injury. In some embodiments, such parameter may be hospitalization-related, e.g., length of hospital stay, length of intensive care unit (ICU) stay, number of days on a ventilator, and/or disposition from hospital.

In some embodiments, clinical parameters may include, e.g., biological fluids and/or tissues isolated from a subject or patient, which can be tested by the methods of the present disclosure described herein, and include but are not limited to whole blood, peripheral blood, serum, plasma, cerebrospinal fluid, wound effluent, urine, amniotic fluid, peritoneal fluid, pleural fluid, lymph fluids, various external secretions of the respiratory, intestinal, and genitourinary tracts, tears, saliva, white blood cells, solid tumors, lymphomas, leukemias, myelomas, and combinations thereof. In some embodiments, the clinical parameters are one or more of biomarkers, administration of blood products, and injury severity scores.

In some embodiments, at a step 204, the method may include preprocessing the plurality of medical parameters to determine a plurality of medical features. For example, system 100 (FIG. 1) may preprocess the plurality of medical parameters to determine a plurality of preprocessed medical features.

In some embodiments, a preprocessing stage may include data preparation. Data preparation may include cleaning data, transforming data, and/or selecting subsets of records. In some embodiments, data preparation can include executing pre-processing operations on the data. For example, an imputation algorithm can be executed to generate values for missing data. Up-sampling and/or predictor rank transformation can be executed (e.g., for feature selection) to accommodate class imbalance and non-normality in the data.

In some embodiments, executing the imputation algorithm includes interpolating or estimating values for the missing data, such as by generating a distribution of available data for a clinical parameter having missing data, and interpolating values for the missing data based on the distribution.

In some embodiments, a data cleaning step may be configured to define plausible limits for vital signs, e.g., temperature, HR, blood pressure and/or automatically exclude implausible values.

In some embodiments, a time handling step may be configured to generate a time-dependent representation of one or more parameters using, for example, a Fourier transform, polynomial adjustments, and/or various statistical tools. In some embodiments, the time handling step may include automatically and/or manually combining a plurality of medical samples and/or measurements taken from a patient over a sequence of time periods to determine and/or create a at least one combined parameter and/or feature which may represent patterns of change of the plurality of medical samples over time and/or time-series variables. The combined parameter may be added to the dataset.

In some embodiments, a feature extraction step may be configured to generate additional features, e.g., based on relations between existing features in the dataset, and add the additional features to the dataset.

In some embodiments, a step 206 may be configured to perform a feature selection stage, to, e.g., identify the most relevant variables and predictors from the set of parameters obtained in step 202.

In some embodiments, variable and/or feature selection can include executing supervised machine learning algorithms, such as constraint-based algorithms, constraint-based structure learning algorithms, and/or constraint-based local discovery learning algorithms.

In some embodiments, feature selection can be executed to identify a subset of variables in the training data which have desired predictive ability relative to a remainder of the variables in the training data, enabling more efficient and accurate predictions using a model generated based on the selected variables. In some embodiments, feature selection is performed using machine learning algorithms, e.g., a boosting ensemble such as XGBoost, Grow-Shrink (“gs”), Incremental Association Markov Blanket (“iamb”), Fast Incremental Association (“fast, iamb”), Max-Min Parents & Children (“mmpc”), or Semi-Interleaved Hiton-PC (“si.hiton.pc”) algorithms. However, various other implementations of such machine learning algorithms may be used to perform feature selection and other processes described herein. In some embodiments, feature selection can search for a smaller dimension set of variables that seek to represent the underlying distribution of the full set of variables, which attempts to increase generalizability to other data sets from the same distribution.

In some embodiments, feature selection may be performed by removing variables that are highly correlated. Several algorithms can be used to search the input dataset with ranked predictors to find a reduced variable set that best represented the underlying distribution of all variables with respect to the infectious complication outcomes. A feature selection filter algorithm can be used to choose the reduced variable set.

For example, in some embodiments, one or more of the Maximum Minimum Parents Children (mmpc) and/or the inter.iamb algorithm can be used to choose the nodes of the corresponding Bayesian network as the reduced variable set.

In some embodiments, feature selection is performed to search the training data for a subset of variables which are used as nodes of Bayesian networks. A Bayesian network (e.g., belief network, Bayesian belief network) is a probabilistic model representing a set of variables and their conditional dependencies using a directed acyclic graph. For example, in the context of diagnostic prediction, feature selection can be used to select variables from the training data to be used as nodes of the Bayesian network; given values for the nodes for a specific subject, a prediction of a diagnosis for the subject can then be generated.

In some embodiments, the prediction module is trained on a dataset generated though feature selection performed by, e.g., feature selection module 110b to select a subset of model parameters from the plurality of clinical parameters. The feature selection can be used to identify biological effector and non-biological effector components that are critical to the BSI outcomes. In some embodiments, the prediction module 110c can execute classification on the selected model parameters to select a candidate model for generating BSI outcome/risk predictions.

In some embodiments, a step 208 may include generating a training dataset for a machine learning classification model, based, at least in part, on the collected parameters and the feature selection process performed by, e.g., feature selection module 110b.

In some embodiments, the training dataset comprises values of clinical parameters associated with BSI outcomes in subjects. The values of the clinical parameters can be received and stored for each of a plurality of subjects. The training dataset can receive and store values of at least one clinical parameter of a plurality of clinical parameters and a corresponding BSI outcome. The training dataset can associate the values of the plurality of clinical parameters to the corresponding BSI outcome for each of the plurality of subjects. In some embodiments, the training dataset stores values of the plurality of clinical parameters that are associated, for each subject, with a single point in time.

As noted above, the clinical parameters can include at least some of gender, age, date of injury, location of injury, presence of abdominal injury, mechanism of injury, wound depth, wound surface area, associated injuries, type of wound closure, success of wound closure, requirement for transfusion, total number of blood products transfused, amount of whole blood cells administered to the subject, presence of traumatic brain injury, severity of traumatic brain injury, length of hospital stay, length of intensive care unit (ICU) stay, number of days on a ventilator, disposition from hospital, development of nosocomial infections, and the like.

The BSI outcome can be based on presence of bacteria in the blood such as may be diagnosed through isolation of a pathogen from at least one quantitated blood culture. In some embodiments, a pathogen is isolated from at least two blood cultures. The BSI outcome may be a binary variable (e.g., BSI is present in the first subject or BSI is not present in the first subject).

In some embodiments, at a step 210, a machine learning classifier of the present disclosure, e.g., prediction module 110c, is trained on the training dataset to generate a classification model. In some embodiments, prediction module 110c can generate models for predicting BSI outcomes (and risks thereof) which use a reduced set of clinical parameters as variables.

The prediction module 110c can execute classification algorithms (e.g., binary classification algorithms) for each subset of model parameters to generate predictions of BSI outcomes based on the subsets of model parameters. In some embodiments, the prediction module 110c executes classification algorithms including but not limited to linear discriminant analysis (lda), classification and regression trees (cart), It-nearest neighbors (knn), support vector machine (svm), logistic regression (glm), random forest (rf), generalized linear models (glmnet), and/or naive Bayes (nb). In some embodiments, classification may be defined as the task of generalizing a known structure to be applied to new data. Classification algorithms can include linear discriminant analysis, classification and regression trees/decision tree learning/random forest modeling, nearest neighbor, support vector machine, logistic regression, generalized linear models, Naive Bayesian classification, and neural networks, among others.

In some embodiments, executing a random forest model classification algorithm can include generating a plurality of decision trees using the training dataset. Each decision tree may be generated by, e.g., bootstrap aggregating with replacement the first values of the plurality of clinical parameters in the training dataset. The decision trees may be generated to make decisions using the subset of model parameters.

To generate predictions of BSI outcomes, prediction module 110c can use test values for the model parameters as inputs in the random forest model classification algorithm. For example, a decision tree can include a hierarchical organization of nodes, including terminal nodes where, based on the decision made, the decision tree can output a prediction of a BSI outcome (e.g., an indication that the subject has BSI or that the subject is well). A random forest model classification algorithm can then count the number of BSI outcomes (e.g., BSI vs. Well) calculated by each decision tree, and output the predicted BSI outcomes based on the counts. For example, the random forest model classification algorithm can compare the count of “BSI” outputs to the count of “Well” outputs and output the predicted BSI outcome to indicate that the subject is predicted to have BSI responsive to the count of BSI outputs being greater than the count of Well outputs (or vice versa). The random forest classification algorithm can also output the prediction of the BSI outcome as a probability based on the number of BSI outcomes: for example, if the random forest model includes 10,000 decision trees, of which 5,000 indicate a BSI outcome, the random forest model classification algorithm can output the prediction of BSI outcome as a probability of 50%.

In some embodiments, a trained machine learning classification model of the present disclosure, as generated in step 210, can include, e.g., cluster analysis, regression (e.g., linear and non-linear), classification, decision analysis, and/or time series analysis, among others.

In some embodiments, the number of decision trees used may be several hundred trees, which can improve computational performance of the machine learning systems by reducing the number of calculations needed to execute the random forest model. In some embodiments, each random forest decision tree is generated by bootstrap aggregating (“bagging”), where for each decision tree, the training data is randomly sampled with replacement to generate a randomly sampled set of training data, and then the decision tree is trained on the randomly sampled set of training data. In some embodiments, where feature selection is performed prior to generated the random forest model, the training data is sampled based on the reduced set of variables from feature selection (as opposed to sampling based on all variables).

In some embodiments, in order to perform a prediction given values of variables for a subject, each decision tree is traversed using the given values until a decision rule is reached that is followed by terminal nodes (e.g., presence of disease in the subject, no presence of disease in the subject). The outcome from the decision rule followed by the terminal nodes is then used as the outcome for the decision tree. The outcomes across all decision trees in the random forest model are summed to generate a prediction regarding the subject.

In some embodiments, the machine learning training stage may include training an ensemble of machine learning models, e.g., including six random forest models and/or two XGBoost models. In other embodiments, any other combination of random forest models and XGBoost models may be trained. In some embodiments, the ensemble of machine learning models may be trained periodically and/or only one time.

In some embodiments, the ensemble of machine learning models may be trained to analyze and/or determine a probability of BSI, e.g., by running each model of the ensemble independently, to determine for each model an independent prediction, and then selecting and/or choosing a final prediction based on all independent predictions.

In some embodiments, the ensemble of machine learning models may be configured to determine the final prediction based on a soft voting method, for example, which may include averaging probabilities received from each machine learning model, e.g., the independent predictions.

In other embodiments, the ensemble of machine learning models may determine the final prediction based on any other criteria and/or method, e.g., a hard voting method.

In some embodiments, utilizing the ensemble of machine learning models may provide a more reliable and/or accurate result, for example, at least compared to utilizing one of the machine learning models alone. For example, an ensemble of machine learning models including six random forest models and two XGBoost models may receive medical parameters of a patient. The six random forest models may output six corresponding probabilities of an infection based on the medical parameters, and the two XGBoost models may output two corresponding probabilities of the infection based on the medical parameters. All probabilities of the infection may then be averaged to provide an optimal and/or accurate prediction for the patient.

In some embodiments, after classifier training stage, the ensemble of machine learning models may be configured to implement a validation process, e.g., through a first evaluation which may include, e.g., a cross-validation. The cross validation may be configured to randomly divide the training set into, e.g., ten folds. The ten-fold validation may then run ten times, for example, using nine different folds of the training set for machine learning modeling, and a tenth fold for validation. The results may be assessed through a computation of statistical measures, e.g., average and a confidence interval of an Area Under a Receiver Operating Characteristic curve (AUROC) for the ten different evaluation folds. In some embodiments, a second evaluation may include an assessment of a machine learning model on a validation set, e.g., the tenth fold for validation which may include 10% of the original data. In some embodiments, a third evaluation may include a statistical analysis, for example, including presenting population characteristics by median and InterQuartile Range (IQR) for skewed data, and a mean with standard deviation for normal distributed data, e.g., using bootstrapping techniques.

In some embodiments, a cross validation process of the machine learning model may implement a statistical method configured to estimate a skill of a machine learning model on a limited data sample, e.g., in order to estimate how the machine learning model is expected to perform when used to make predictions on data which was not used when training the machine learning model.

In some embodiments, the cross validation process of the machine learning model may include splitting a given data sample into a plurality of groups and/or folds, for example, ten groups and/or folds.

In some embodiments, the validation process may be implemented after every training stage, e.g., which may be periodically and/or only one time.

In some embodiments, the BSI prediction algorithm may be configured to preprocess data and/or information of the dataset, e.g., as discussed with respect to the infection prediction algorithm.

In some embodiments, features which are found to be most predictive for separating BSI and non-BSI scenarios and/or episodes, may not necessarily be predictive by themselves, but rather may contribute to an ability of the BSI prediction algorithm to differentiate BSI from non-BSI scenarios episodes, e.g., when combined with other selected features.

In some embodiments, the BSI prediction algorithm may input selected features, e.g., the 50 selected features from the feature selection step, to the machine learning prediction model, e.g., including six random forest models and/or two XGBoost models.

In some embodiments, the machine learning modeling step may be trained to analyze a probability of BSI as assessed by different models, e.g., six random forest models and/or two XGBoost models, and by the soft voting method, e.g., configured to average probabilities received from each random forest and/or XGBoost model.

In some embodiments, the validation process may include a statistical analysis including an assessment of a difference between BSI and non-BSI groups in the demographic data, in background clinical parameters, and in recorded clinical parameters of both datasets. The assessment may be evaluated with an χ2 test for categorical variables, and with Welch's unequal variances t-test for continuous numeric variables. A CI of cross-validation and test-set validation may be implemented and/or performed with bootstrapping.

In some embodiments, the validation process may be implemented by a cross-validation method and a test-set validation method.

In some embodiments, at a step 212, a trained prediction module 110c can be applied, at an inference stage, to predict a BSI outcome specific to at least one target patient and/or subject. In some embodiments, prediction module 110c can receive, for the at least one target patient, values associated with at least one clinical parameter of the plurality of clinical parameters.

In some embodiments, at least one of the received values corresponds to a model parameter of the subset of model parameters used in the classification algorithm. If prediction module 110c receives several values of clinical parameters, of which at least one does not correspond to a model parameter of the subset of model parameters, prediction module 110c may execute an imputation algorithm to generate a value for such a missing parameter.

In some embodiments, system 100 can update the training dataset based on the values received for the target patient, as well as the predicted BSI outcomes. As such, system 100 continually learn from new data regarding subjects. System 100 can store the predicted BSI outcome with an association to the value(s) received for the target patient in the training dataset. The predicted BSI outcome may be stored with an indication of being a predicted value (as compared to the known BSI outcomes for the plurality of first subjects). Over time, system 100 may also store the known BSI outcome with an indication of an update relative to the predicted BSI outcome.

### Experimental Results

The present BSI detection algorithm was tested on datasets obtained from the following two medical centers:
The 18-bed surgical-medical ICU of Rambam Healthcare Campus (RHCC), Haifa, Israel between January 2013 and December 2017; and
the surgical, trauma-surgical, and medical ICUs of Beth Israel Deaconess medical center (BIDMC), Boston, Mass. between January 2008 and December 2012.

The data of RHCC was gathered from two computerized database systems. “iMD soft Metavision,” is the database software of the hospital's ICU which records vital signs, laboratory measures, fluid balance, dosage and duration of all pharmacologic treatments, the time of insertion and withdrawing of various invasive devices. “Prometheus” is the hospital's electronic patient file containing demographic information, underlying medical conditions, chronic medications, the timing of surgeries and procedures during the admission and more comprehensive laboratory results, including microbiologic data.

The data of BIDMC was gathered from the MIMIC III database, research purpose database which comprise the information that was gathered from the ICU and the hospital electronic files. The database includes demographic details, vital signs, laboratory measurements, fluid balance, dosage and duration of all pharmacologic treatments and registry of all fatalities. It does not comprise the chronic medication treatment and the timing of surgeries performed during an admission. The medical diagnoses list is available only at discharge time.

The study population included all patients for whom blood cultures (BC) were collected for suspected bacteremia at least 48 hours after admission to the ICU. The sampling time was defined as the time of the BC collection, as recorded in the microbiologic file. For patients with multiple positive blood cultures, we included only the first episode. For patients with no positive BC, we randomly selected one of the (negative) blood cultures as the index event.

The outcome assessed was BSI, defined as growth of any pathogen in at least one blood culture bottle. The growth of pathogens considered likely to represent contamination rather than true infection was not considered as bacteremia. These pathogens included coagulase-negative staphylococci, Corynebacterium species, Bacillus species, Diphteroides, Aerococcus, and Propionibacterium.

During the study period, 1812 patients were hospitalized in the general ICU of RHCC for more than two days. Single or multiple blood cultures (BC) for a suspected ICU-acquired infection were collected for 1,021/1,812 (56.3%) of all patients. Among those, 162 patients (8.9%) had an ICU-acquired BSI.

In BIDMC ICUs, 7419 patients were admitted for more than two days during the study period. Single or multiple BC for a suspected ICU-acquired infection were collected for 2351/7419 (31.7%). Among those, 151 patients had an ICU-acquired BSI (6.4%).

The demographic and the background medical diagnoses of the study populations are shown in Table 1:

| Characteristics | RHCC | | BIDMC | |
|---|---|---|---|---|
| Median age at | 56.2 | (38.5-68.8) | 62 | (51-75) |
| admission (IQR), y | | | | |
| Surgical patients, | 646 | (63.3) | 983 | (41.8) |
| no. (%) | | | | |
| Male gender | 684 | (67) | 1333 | (56.7) |
| Diabetes | 277 | (27.1) | 681 | (29) |
| Chronic renal | 117 | (11.5) | 541 | (20.3) |
| failure | | | | |
| Ischemic heart | 170 | (16.7) | 259 | (11) |
| disease | | | | |
| Heart failure | 119 | (11.7) | 564 | (24) |
| COPD | 122 | (12) | 127 | (5.4) |
| Asthma | 39 | (3.8) | 167 | (7.1) |
| Hematologic | 37 | (3.6) | 148 | (6.3) |
| malignancy | | | | |
| Solid neoplasm | 112 | (11) | 258 | (11) |
| Connective tissue | 15 | (1.5) | 87 | (3.7%) |
| disease | | | | |
| Liver cirrhosis | 20 | (2) | 259 | (11) |
| CVA | 67 | (6.6) | 258 | (11) |
| Dementia | 4 | (0.4) | 9 | (2.6) |
| Burns | 46 | (4.5) | N/A | |
| (above 30% TBCA) | | | | |
| HIV | 2 | (0.2) | 24 | (1) |
| Immunosuppressant | 88 | (8.6) | N/A | |
| treatment | | | | |
| Were ventilated at | 801/1021 | (78.4%) | 1585/2351 | (67.4%) |
| admission day | | | | |
| Were treated with | 423/1021 | (41.4%) | 862/2351 | (36.6%) |
| vasopressors at | | | | |
| admission day | | | | |

30-day mortality rates in both institutions, calculated from the day of blood culture sampling, were higher for patients with BSI than for patients without BSI in Table 2:

| Medical | Mortality Rate - BSI | | Mortality Rate - BSI | | |
|---|---|---|---|---|---|
| center | Negative (patients) | | Positive (patients) | | p-value |
| RHCC | 21.6% | (186) | 31.5% | (51) | 0.007 |
| BIDMC | 26.7% | (589) | 44.3% | (67) | <0.001 |

BIDMC and RHCC datasets were separated into training and validation sets. The training sets which used for learning and cross-validation purpose, comprise 90% of the data: 2166 and 918 patients for BIDMC and RHCC respectively. The validation dataset comprised of 10% of the data: 235 and 103 patients for BIDMC and RHCC respectively.

In RHCC and BIDMC, the data set included 6400 and 7500 different features, respectively, for each patient. Most of these features represented patterns of change in the time-series variables. The other features were: the clinical status at the sampling date (last values of laboratory and vital signs measurements), background demographic data, background clinical information (for RHCC only), placement of different indwelling catheters and the duration of their use in relation to the sampling time, antibiotic treatment, dialysis, TPN use, and surgical procedures performed prior to sampling time in RHCC. BIDMC dataset included more features per patient because of a larger amount of laboratory measures available for modeling.

For both study groups, the feature selection algorithm found the 50 features that were the most predictive of BSI. Only these selected features were used by the model.

Features selected for the prediction model were: Patterns of change in the time series variables (laboratory and vital signs) and the length of stay in the ICU and the hospital. For RHCC, where the background medical diagnoses were available for modeling, none of these features were included.

The ten features found to be the most predictive for separating BSI and non-BSI episodes are shown in:
RHCC
   Maximum value of BUN (mg/dl) as measured during the 5 days prior sampling
   Last value of MAP (mmHg) minus the median MAP during the 5 days prior sampling
   Y-axis intercept of the linear regression of the Direct bilirubin (μmol/L) during the 5 days prior sampling
   Last Total bilirubin (μmol/L) minus the median total bilirubin at the 3 days prior sampling
   Main frequency of DBP (mmHg) during the 3 days prior sampling (derived from the fast Fourier transform)
   Time duration (hours) between the sampling time and the minimum DBP during the 3 days prior sampling
   Second derivation of the slope of the BUN during the 5 days prior sampling
   Time duration (hours) between the sampling and the hospital admission time
   Median temperature (Celsius) during the 5 days prior sampling
   Time duration (hours) between sampling date and the time of the minimal neutrophils count during the 5 days prior sampling
BIDMC
   The slope of the linear regression of BUN (mg/dl) as measured during the 3 days prior sampling
   Y-axis intercept of the linear regression of the pulse-oxymetry O2 saturation (%) as measured during the 3 days before sampling
   Y-axis intercept of the linear regression of the DBP during the days prior sampling
   Last Total bilirubin (μmol/L) minus the median total bilirubin at the 3 days prior sampling
   Time duration (hours) between the sampling time and the first minimum lymphocyte count during the 3 days prior sampling
   Time duration (hours) between the sampling time and the minimum Anion-gap during the 3 days prior sampling
   Second derivation of the linear regression of PaO2 (mmHg) during the 3 days prior sampling
   Time duration (hours) between the sampling and the hospital admission time
Mean MAP (mmHg) during the 3 days prior sampling
   Time duration (hours) between sampling time and the time of the minimum temperature (Fahrenheit) during the 3 days prior sampling.

In some embodiments, additional and/or other clinical parameters may be considered as primary predictors in the case of BSI, including, but not limited to:
Gastro-intestinal function parameters:
   Defecation frequency during the 24, 48, 72 and 96 hours prior to the index time for outcome prediction,
   total time without defecations during these periods,
   vomiting frequency,
   evidence of the amount of gastric residual volume,
   gastric and intestinal acidity, and/or
   intra-abdominal pressure (IAP).

These features were not necessarily predictive by themselves, but rather contributed, combined with other included features, to the ability of the prediction model to differentiate BSI from non-BSI episodes.

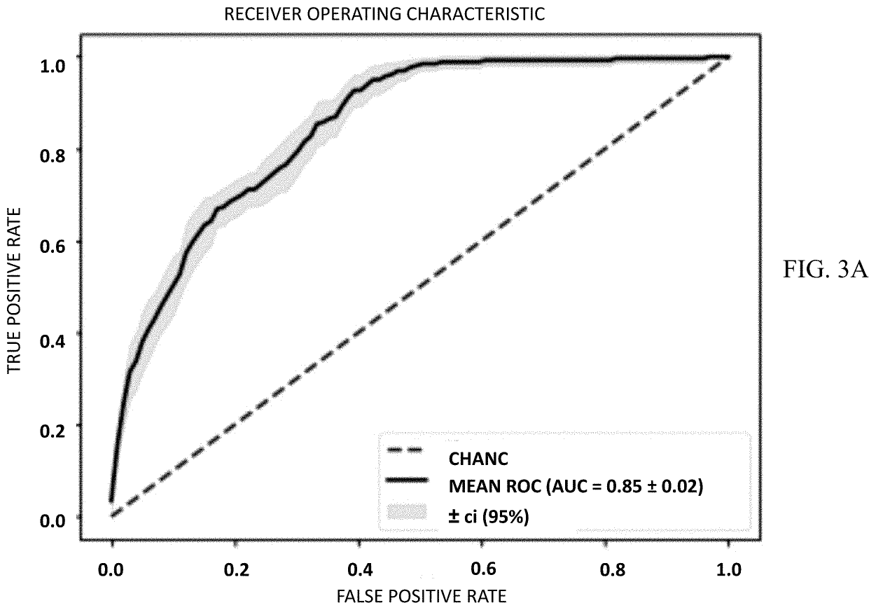

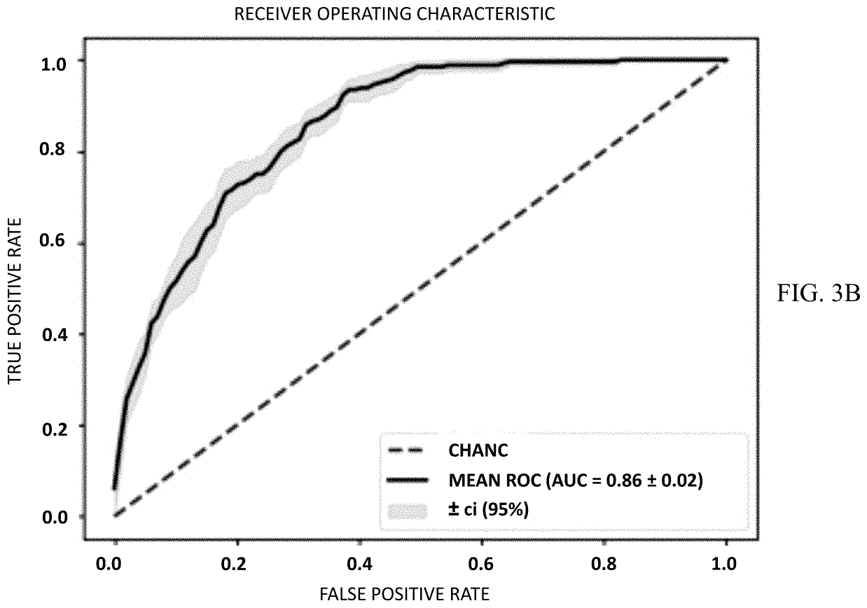

The evaluation of the model's prediction ability was performed by cross-validation method and by test-set validation. BIDMC cross-validation AUROC was 0.86, with 95% CI of ±0.02, the test set AUROC was 0.85 with 95% CI of ±0.02. The results are displayed in FIGS. 3A-3B.

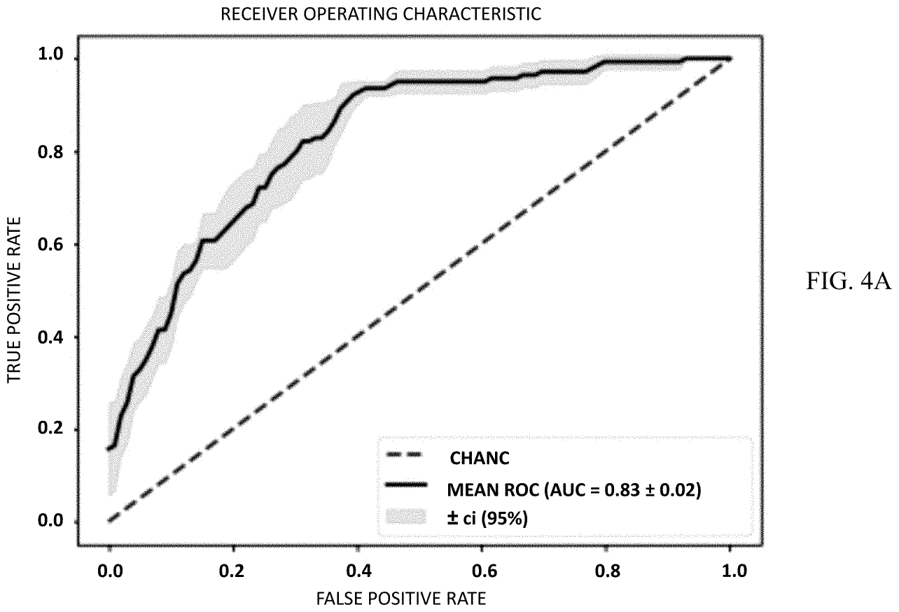

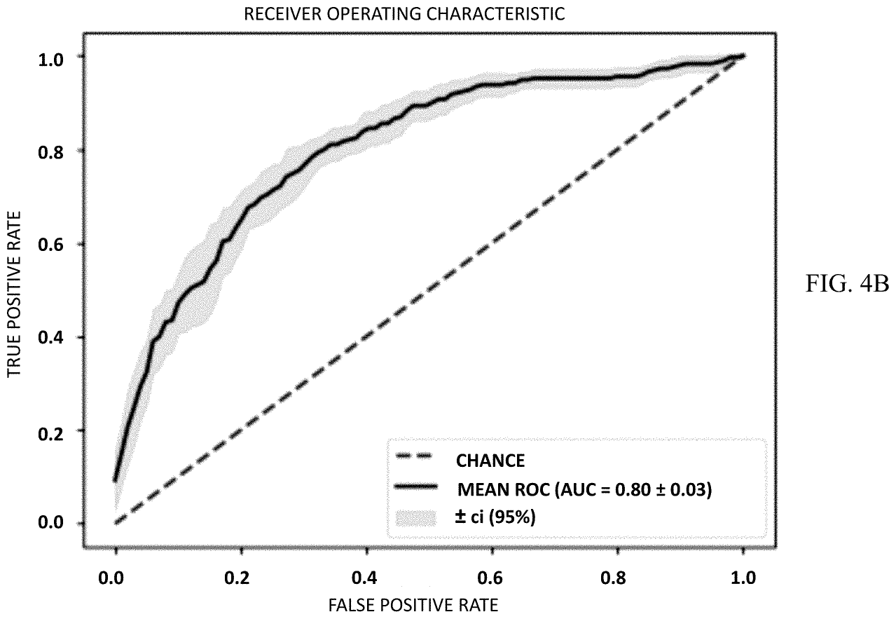

RHCC AUROC for cross-validation and test-set validation were 0.83 with 95% CI of ±0.02 and 0.80 95% CI of ±0.03, respectively. The results are displayed in FIGS. 4A-4B.

The results at both BIDMC and RHCC indicate that it is possible to identify patients at high risk of BSI from 24 to 72 hours before blood cultures become positive, by using readily available data, e.g., which may be independent of any subjective clinical judgment and/or cost efficient.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may include copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of modified purpose computer, special purpose computer, a general computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A system for predicting a medical condition in a patient, the system comprising:
at least one hardware processor; and
a non-transitory computer-readable storage medium having stored thereon program instructions, the program instructions executable by the at least one hardware processor to:
receive, with respect to each of a plurality of subjects, a plurality of clinical parameters, and an outcome indication with respect the said medical condition,
apply to said plurality of clinical parameters one or more feature selection algorithms, to select a subset of said plurality of clinical parameters as the most relevant predictors,
at a training stage, train a machine learning model on a training set comprising:
(i) said relevant predictors with respect to each of said subjects, and
(ii) labels associated with said outcome indication in said subject, and
at an inference stage, apply said trained machine learning model to a target subset of said relevant predictors with respect to a target patient, to predict said medical condition in said target patient.

2. The system of claim 1, wherein said medical condition is a bloodstream infection (BSI), and said relevant predictors are selected from the group consisting of: blood urea nitrogen parameters, mean arterial pressure parameters, bilirubin parameters, blood pressure parameters, hospitalization duration parameters, body temperature parameters, neutrophils count parameters, blood oxygen saturation parameters, lymphocyte count parameters, anion gap parameters, and partial pressure of oxygen parameters.

3. The system of claim 1, wherein said medical condition is extubation failure risk and said relevant predictors are selected from the group consisting of: sedative drug dosage parameters prior to extubation, mean alveolar pressure parameters, hemodynamic parameters, respiratory parameters, heart rate parameters, respiratory rate parameters, and arterial blood pressure parameters.

4. The system of claim 1, wherein said medical condition is mortality risk within a specified time period, and said relevant predictors are selected from the group consisting of: hemodynamic parameters, respiratory parameters, heart rate parameters, respiratory rate parameters, and arterial blood pressure parameters, patient medical history, bilirubin parameters, hemoglobin parameters, red blood cell indices, glucose parameters, creatinine parameters, and albumin parameters.

5. The system of claim 4, wherein said patient medical history comprises prior medical diagnoses of at least some of: ischemic heart disease, congestive heart failure, chronic obstructive pulmonary disease, chronic renal failure, end-stage renal disease, diabetes without target organ damage, diabetes with organ damage, acute leukemia, chronic leukemia, lymphoma, multiple myeloma, human immunodeficiency virus infection, malignancy, cirrhosis, cerebral vascular accident, transient ischemic attack, and dementia.

6. The system of claim 1, wherein said relevant predictors further include gastro-intestinal function parameters selected from the group consisting of: defecation frequency during the preceding 24, 48, 72 and 96 hour periods; total time without defecations during the preceding 24, 48, 72 and 96 hour periods; vomiting frequency; evidence of the amount of gastric residual volume; gastric and intestinal acidity; and intra-abdominal pressure (IAP).

7. The system of claim 1, wherein said plurality of clinical parameters further comprises clinical data monitored in connection with hospital admission selected from the group consisting of: body temperature; hemodynamic and respiratory parameters; heart rate; systolic blood pressure; diastolic blood pressure; mean arterial pressure; urine output; respiratory rate; pulse oximetry O2 saturation; timing, duration, and dosage of intravenous fluids; diuretics; vasopressor; antibiotic treatment; total parenteral nutrition; enteral nutrition; continuous renal replacement therapy and dialysis; presence and timing of indwelling catheters; surgeries during admission; duration of hospitalization and ICU stay; use of glucocorticoids; and chemotherapy.

8. The system of claim 1, wherein said feature selection algorithms comprise at least an extreme gradient boosting algorithm.

9. The system of claim 1, wherein said machine learning model comprises a plurality of classification algorithms selected from the group consisting of: linear discriminant analysis (Ida), classification and regression trees (cart), It-nearest neighbors (knn), support vector machine (svm), logistic regression (glm), random forest (if), generalized linear models (glmnet), naive Bayes (nb), and extreme gradient boosting.

10. The system of claim 9, wherein said applying comprises applying each of said plurality of classification algorithms to said target subset to obtain a plurality of corresponding predictions, and wherein a final prediction of said medical condition is based, at least in part, on a weighted soft voting of all of said plurality of predictions, and wherein said soft voting is based, at least in part, on a confidence score associated with each of said plurality of predictions.

11. (canceled)

12. A method for predicting a medical condition in a patient, the method comprising:
receiving, with respect to each of a plurality of subjects, a plurality of clinical parameters, and an outcome indication with respect the said medical condition;
applying to said plurality of clinical parameters one or more feature selection algorithms, to select a subset of said plurality of clinical parameters as the most relevant predictors;
at a training stage, training a machine learning model on a training set comprising:
(i) said relevant predictors with respect to each of said subjects, and
(ii) labels associated with said outcome indication in said subject; and
at an inference stage, applying said trained machine learning model to a target subset of said relevant predictors with respect to a target patient, to predict said medical condition in said target patient.

13. The method of claim 12, wherein said medical condition is a bloodstream infection (BSI), and said relevant predictors are selected from the group consisting of: blood urea nitrogen parameters, mean arterial pressure parameters, bilirubin parameters, blood pressure parameters, hospitalization duration parameters, body temperature parameters, neutrophils count parameters, blood oxygen saturation parameters, lymphocyte count parameters, anion gap parameters, and partial pressure of oxygen parameters.

14. The method of claim 12, wherein said medical condition is extubation failure risk and said relevant predictors are selected from the group consisting of: sedative drug dosage parameters prior to extubation, mean alveolar pressure parameters, hemodynamic parameters, respiratory parameters, heart rate parameters, respiratory rate parameters, and arterial blood pressure parameters.

15. The method of claim 12, wherein said medical condition is mortality risk within a specified time period, and said relevant predictors are selected from the group consisting of:
hemodynamic parameters, respiratory parameters, heart rate parameters, respiratory rate parameters, and arterial blood pressure parameters, patient medical history, bilirubin parameters, hemoglobin parameters, red blood cell indices, glucose parameters, creatinine parameters, and albumin parameters.

16. The method of claim 15, wherein said patient medical history comprises prior medical diagnoses of at least some of: ischemic heart disease, congestive heart failure, chronic obstructive pulmonary disease, chronic renal failure, end-stage renal disease, diabetes without target organ damage, diabetes with organ damage, acute leukemia, chronic leukemia, lymphoma, multiple myeloma, human immunodeficiency virus infection, malignancy, cirrhosis, cerebral vascular accident, transient ischemic attack, and dementia.

17. The method of claim 12, wherein said relevant predictors further include gastro-intestinal function parameters selected from the group consisting of: defecation frequency during the preceding 24, 48, 72 and 96 hour periods; total time without defecations during the preceding 24, 48, 72 and 96 hour periods; vomiting frequency; evidence of the amount of gastric residual volume; gastric and intestinal acidity; and intra-abdominal pressure (IAP).

18. The method of claim 12, wherein said plurality of clinical parameters further comprises clinical data monitored in connection with hospital admission selected from the group consisting of: body temperature; hemodynamic and respiratory parameters; heart rate; systolic blood pressure; diastolic blood pressure; mean arterial pressure; urine output; respiratory rate; pulse oximetry O2 saturation; timing, duration, and dosage of intravenous fluids; diuretics; vasopressor; antibiotic treatment; total parenteral nutrition; enteral nutrition; continuous renal replacement therapy and dialysis; presence and timing of indwelling catheters; surgeries during admission; duration of hospitalization and ICU stay; use of glucocorticoids; and chemotherapy.

19. The method of claim 12, wherein said feature selection algorithms comprise at least an extreme gradient boosting algorithm.

20. The method of claim 12, wherein said machine learning model comprises a plurality of classification algorithms selected from the group consisting of: linear discriminant analysis (Ida), classification and regression trees (cart), It-nearest neighbors (knn), support vector machine (svm), logistic regression (glm), random forest (if), generalized linear models (glmnet), naive Bayes (nb), and extreme gradient boosting.

21. The method of claim 20, wherein said applying comprises applying each of said plurality of classification algorithms to said target subset to obtain a plurality of corresponding predictions, and wherein a final prediction of said medical condition is based, at least in part, on a weighted soft voting of all of said plurality of predictions, and wherein said soft voting is based, at least in part, on a confidence score associated with each of said plurality of predictions.

22.-33. (canceled)
